(19) United States
(12) Patent Application Publication
(10) Pub. No.: US 2010/0099099 A1
(43) Pub. Date: Apr. 22, 2010

(54) METHOD AND TEST KIT FOR THE RAPID DETECTION OF SPECIFIC NUCLEIC ACID SEQUENCES, ESPECIALLY FOR DETECTING OF MUTATIONS OR SNPs

(75) Inventors: Elmara Graser, Berlin (DE); Timo Hillebrand, Hoenow (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: AJ INNUSCREEN GmbH, Berlin (DE)

(21) Appl. No.: 12/559,252

(22) Filed: Sep. 14, 2009

Related U.S. Application Data
(63) Continuation of application No. PCT/EP08/53065, filed on Mar. 14, 2008.

(30) Foreign Application Priority Data
Mar. 14, 2007 (DE) 10 2007 013 099.8

(51) Int. Cl. C12Q 1/68

(52) U.S. Cl. 435/6

(57) ABSTRACT

A method and a test kit for rapid detection of specific nucleic acid sequences, especially for detection of mutations or single nucleotide polymorphisms (SNPs), in which the detection reaction takes place in two steps. The first step involves the target-specific amplification reaction, coupled with the probe-hybridization reaction using fluorescence-labeled allele-specific amplification primers. In the second step, the fluorescence is detected by means of commercial fluorescence readers. Genotyping is carried out from the ratio of the end-point fluorescence of the samples and negative controls.

### CROSS REFERENCE TO RELATED APPLICATIONS

This application is a continuation of International Application No. PCT/EP08/053,065, filed Mar. 14, 2008. Priority is claimed to GERMANY 10 2007 013 099.8, filed Mar. 14, 2007. Both of these documents are incorporated by reference in their entireties.

### BACKGROUND OF THE INVENTION

1. Field of the Invention

The invention relates to a method and a test kit for rapid detection of specific nucleic acid sequences, especially for detection of mutations or single nucleotide polymorphisms (SNPs), wherein the detection reaction takes place in two steps. The first step involves the target-specific amplification reaction, coupled with the probe-hybridization reaction using fluorescence-labeled allele-specific amplification primers. This reaction can be carried out on all commercial PCR (polymerase chain reaction) instruments and is not limited to real-time PCR instruments. In the second step, the fluorescence is detected by means of commercial fluorescence readers.

2. Description of the Related Art

Genetic diagnostics has become an indispensable tool of modern laboratory diagnostics and fundamental research in medicine. Association with the at-risk groups of metabolic diseases, hematopoietic diseases or oncological diseases as well as with therapeutic recommendations for such diseases are only some examples of applied mutation diagnostics. Allelic variation in humans and other organisms and numerous single nucleotide polymorphisms defining different alleles are well known, Lo, et al., Genome Research, Allelic Variation in Gene Expression Is Common in the Human Genome (2003). Genotyping provides a measurement of the genetic variation between members of a species. Single nucleotide polymorphisms (SNP) represent a common type of genetic variation. A SNP is a single base pair mutation at a specific genetic locus, usually consisting of two different alleles. SNPs are of increasing interest for evaluating disease risk, resistance or susceptibility, in conjunction with pharmacogenetics, or for the evaluation of personal traits or genealogy.

Over the past years, numerous methods capable of detecting changed nucleic acid sequences, especially in the case of single-base changes or for SNPs, have been developed for this purpose.

A simple and inexpensive method for detection of specific nucleic acid sequences and of mutative changes of the DNA sequence is RFLP analysis (restriction fragment length polymorphism). A DNA fragment containing the mutation site is amplified. Then the PCR product is digested with an endonuclease, which has its recognition sequence exactly at the mutation site. Detection is achieved by electrophoresis. However, the method cannot be automated and it does not permit parallel processing of large amounts of samples.

The method suffers from the disadvantage that it is not universally usable, since not every mutation site has a matching nuclease recognition site. Furthermore, incomplete or inhibited nuclease digestion leads to false results.

A further relatively inexpensive method for detection of mutations is SSCP analysis (single strand conformation polymorphism). The method is based on the fact that the conformation of single-strand DNA is substantially determined by the base sequence. After amplification of the target sequence to be examined, the generated double-strand PCR product is transformed to the single-strand conformation (for example, by heating or treatment with a denaturing chemical) and then rapidly cooled on ice.

The single-strand DNA is separated by means of native polyacrylamide gel electrophoresis (PAGE). If a mutation is present, the migration behavior in the gel changes. The result is visualized by staining the gel. This method is also suitable in principle for detection of mutations, but is labor-intensive, highly susceptible to errors and has no automation potential.

Further methods for detection of single-base changes are temperature-gradient gel electrophoresis, denaturing-gradient gel electrophoresis or heteroduplex analysis.

All of these methods are traditional techniques that have been and are being used in research laboratories for detection of genetic changes.

A further traditional method for detection of genetic changes is DNA sequencing. This widely used method is a reliable technique for detection of changes of specific nucleotide sequences. It suffers from the disadvantages of being time-consuming and very costly in principle. Instrumental systems capable of high throughputs cost well over 100,000 £. A novel method for rapid detection of changed nucleotide sequences is pyrosequencing. Especially in the field of detection of SNPs, it is possible to process high throughputs of samples with this method. However, this method is also associated with a very expensive instrumental system, in addition to which the chemistry of the reaction is highly complex. Furthermore, as in traditional DNA sequencing, numerous working steps are necessary before the actual detection reaction can be started. This pertains to amplification of the target sequence to be examined, to purification thereof and thereafter to the sequencing reaction.

A further option for detection of single-base changes is PCR-ELISA (enzyme-linked immunosorbent assay). In this method, the DNA sequence to be examined is also amplified again and the generated DNA fragment is then covalently immobilized in a solid phase (such as microtiter plates), denatured to a single strand and hybridized with an allele-specific probe. Successful binding of the probe can be visualized with an antibody-mediated color reaction. However, this method also includes very many experimental working steps and furthermore is susceptible to interference. An advantage of such a method is that the instrumental systems required for it are less expensive than automatic sequencers, for example.

For some years a series of methodological developments has also been taking place in the determination of single-base differences by means of biochip technologies. In this way it should be possible to achieve highly parallel detection of single-base changes on generated small-size DNA patterns (for example, based on previously amplified PCR products) by means of specific hybridization reactions with labeled oligonucleotides. This technology is again extremely expensive and also necessitates working through a large number of reaction steps. Furthermore, extensive experimental expertise is required, and so the tasks cannot be performed in diagnostic routine laboratories. It is also certain that these methods are not suitable for assaying even small numbers of samples efficiently and at an attractive price.

One possibility for detection of single-base differences in a way that in principle can be achieved very rapidly and without great experimental time and effort is real-time PCR methods. In contrast to the methods that have already been briefly described, the detection reaction is carried out in this case as an integrative technique, meaning that the amplification reaction is directly coupled with the actual detection reaction. In this way the needed experimental time and effort can be greatly reduced. Real-time PCR methods are also suitable for examination of single samples and even for examination of samples in the high throughput range.

Such detection of single-base changes by means of real-time PCR can be achieved in various ways. The differences relate on the one hand to the instrumental system used and on the other hand to the use of different probe systems for specific detection of the target sequence.

A widely used method for detection of single-base changes can be achieved by means of light cycler technology (Roche).

For this purpose Roche has developed special hybridization probes, consisting of two different oligonucleotides, each labeled with only fluorochrome. The acceptor is located at the 3′-end of the one probe and the other oligonucleotide has a donor at the 5′-end. The probes are chosen such that they both bind to the same DNA strand, the distance between acceptor and donor being permitted to be at most 1 to 5 nucleotides, so that the FRET effect (fluorescence resonance energy transfer) can occur.

The fluorescence is measured during the annealing step, in which only light of this wavelength is detectable as long as both probes are bound to the DNA. In this system the melting point of both probes should be identical. Because of the use of two hybridizing probes in addition to the primers used, the specificity of this detection system is extremely high. One disadvantage is the difficult probe design. Furthermore, an extremely expensive instrumental system is again necessary to perform the detection reactions.

A further real-time PCR application for detection of single-base changes can be performed with double-dye probes, which are disclosed in U.S. Pat. Nos. 5,210,015 A and 5,487,972 A (TaqMan probes) both of which are hereby incorporated by reference. Double-dye probes carry two fluorochromes on one probe. The reporter dye is located in this case at the 5′-end and the quencher dye at the 3′-end. In addition, a phosphate group is also located at the 3′-end of the probe, so that the probe cannot function as a primer during elongation. As long as the probe is intact, the released light intensity is low, since almost the entire light energy produced after excitation of the reporter is absorbed and transformed by virtue of the spatial proximity of the quencher. The emitted light of the reporter dye is “quenched”, or in other words extinguished. This FRET effect is preserved even after the probe has bonded to the complementary DNA strand. During the elongation phase, the polymerase encounters the probe and hydrolyzes it. The ability of the polymerase to hydrolyze an oligonucleotide (or a probe) during strand synthesis is known as 5′-3′ exonuclease activity. Not all polymerases have 5′-3′ exonuclease activity (Taq and Tth polymerase). This principle was first described for the Taq polymerase. The principle is known as the TaqMan principle. After probe hydrolysis, the reporter dye is no longer located in spatial proximity to the quencher. The emitted fluorescence is now no longer transformed and this fluorescence increase is measured.

For each prepared probe, therefore, what is known as a C_{T} value is determined. The point of intersection between the fluorescence of the sample and the threshold value (background fluorescence) projected onto the abscissa is known as the cycle threshold (C_{T}), and it represents the lowest measurable positive value of the PCR. Thus the C_{T} value represents a number of cycles. It is directly related to the starting amount of DNA used. If the C_{T} value is low, the amount of DNA used is large. If the C_{T} value is high, the starting amount of DNA is low. The C_{T} value is therefore the basis for quantization of a reaction. Genotyping is also possible with this evaluation method, by comparing the C_{T} values of both probes.

A further option for detection of single-base differences by means of real-time PCR technology consists in the use of intercalating dyes (ethidium bromide, Hoechst 33258, Yo-Pro-1 or SYBR Green™ and the like). After being excited by high-energy UV light, these dyes emit light in the lower-energy visible wavelength region (fluorescence). If the dye is present as a free dye in the reaction mixture, the emission is very weak. Only by intercalation of the dye, whereby it fits into the furrows of double-strand DNA molecules, is the light emission greatly intensified. The dyes are inexpensive and universally usable, since in principle any PCR reaction can be followed in real time with them. In addition, they have high signal strength, since every DNA molecule binds several dye molecules. From the advantages, however, there also results an extreme disadvantage for application: in principle it is not possible by means of intercalating dyes to distinguish between correct product and amplification artifacts (such as primer dimers or defective products). While primer dimers and other artifacts are being formed, they naturally also bind intercalating dyes and thus lead to an unspecific increase in fluorescence even in negative samples. However, a clear differentiation between specific amplification event or artifact is absolutely necessary. In order to achieve this in any case, a melting-point analysis is performed at the end of the actual PCR reaction. For this purpose the reaction mixture is heated in steps of 1 degree from 50° C. to 90° C. The fluorescence is measured continuously during this process. The point at which double-strand DNA melts is characterized by a decrease (peak) of the fluorescence of the intercalating dye, since the intercalating dye dissociates from the single-strand DNA. When the PCR is optimally adjusted, a melting-point peak that tapers sharply is to be expected. This melting point represents the specific product to be expected. Products of different sizes and products of other sequences have different melting points. Other methods for determining melting point of nucleic acid duplexes are well-known in the art but are also incorporated by reference to Current Protocols in Molecular Biology, vol. 1 (last updated with supplement 87 in July, 2009), see e.g., units 6.3 and 6.4 and the references cited therein or Human Molecular Genetics, 2^{nd} edition, Wiley-Liss (1999), especially chapters 5 and 6.

When the fluorescence is plotted graphically against temperature, the fluorescence decrease of the two products can be perceived as two separate melting points. Thus this system gains specificity and makes it possible to distinguish a specific amplification product from artifacts. In this way it is possible to distinguish even between homozygotes (single peak) and heterozygotes (two peaks).

As already mentioned and known to those skilled in the art, real-time PCR methods are also suitable for detection of single-base differences in order to solve different types of problems (mutation analysis, SNP genotyping, etc.) and also to achieve specific detection of nucleic acids in general (such as pathogenicity testing). The great advantage of the method consists in the complexity of the analysis, in that the operation of target-specific amplification and detection of features such as single-base differences take place in one reaction vessel. In particular, the real-time methods that function on the basis of using the illustrated different probe systems with combination of reporter and quencher are characterized by a very high degree of diagnostic specificity. For this reason, the methods of real-time PCR have been adopted worldwide for processing of problems in molecular diagnostics and also for detection of mutations or SNPs. What is also common to all of these methods, however, is that they are implemented on very expensive instrumental platforms, which have to unite the process of amplification and that of subsequent optical detection, in a manner corresponding to the problem, in one hardware solution. Furthermore, many of these described detection methods are always based on real-time tracking of the amplification process. On the basis of this strategy, even workup of the measured fluorescence values takes place in the course of the amplification reaction. It is clear to those skilled in the art that an enormously large body of analysis algorithms in real-time systems must also be associated with it. Ultimately this explains the high financial expenditure than must be invested for the use of real-time PCR systems.

The basic object of the invention is therefore to develop a simple, universally usable and inexpensive method capable of detecting specific nucleic acid sequences, and especially to provide a method for detection of mutations or for SNP genotyping.

### BRIEF DESCRIPTION OF THE DRAWINGS

FIG. 1 shows the results of amplification of a human target DNA sequence using the method of the invention.

FIG. 2 compares genotyping using the method of the invention and using DNA sequencing.

### DESCRIPTION OF THE PREFERRED EMBODIMENTS

The object was achieved according to the features described herein. For this purpose the inventive method uses the advantages of the high diagnostic sensitivity and specificity achieved by already established fluorescence probe technologies in the field of real-time PCR methods and the advantage of being able to work on existing instrumental systems that are standard in the laboratory. Furthermore, the previously necessary process of the experimental tasks of molecular sample preparation up to final evaluation of the analysis data is reduced to a minimum.

The inventive method for rapid detection of specific nucleic acid sequences comprises the following steps:

1. Isolation of the nucleic acids.

2. Amplification of the specific target sequence, coupled with the probe-hybridization reaction using fluorescence-labeled allele-specific amplification probes.

3. End-point measurement of the fluorescence without reference dyes to determine the relative size of the reaction fluctuations, and without comparison with already genotyped standard samples.

4. Comparison of the end-point measurement of the fluorescence with negative controls (samples that contain the probes but no DNA).

5. Determination of the genotyping from the ratio of the end-point fluorescence of the probes.

As already described, the real-time PCR methods known from the prior art are always based on continuous measurement and workup of fluorescence signals. Such workup of the continuously measured fluorescence signals requires a highly complex algorithm. The diagnostic result is therefore based on workup of the continuously (in real time) measured fluorescence, not on an end-point fluorescence determination.

An end-point fluorescence measurement is not possible during use of the light cycler technology, since the FRET effect can be measured exclusively during attachment of the probe.

An end-point measurement is possible to only a limited extent even in the use of intercalating dyes, because of their already described lack of specificity of the fluorescence. Furthermore, without the described melting-point measurement, it is not possible to differentiate between different alleles.

In U.S. Pat. No. 6,154,707 B1 there is disclosed a method that permits genotyping of multiple alleles even in the form of end-point measurement of fluorescence after a 5′-exonuclease assay. The method is based on an algorithm that first determines a relative fluorescence by comparison with an internal control and then compares the unknown sample and the sample to be genotyped respectively with a specific control used for one allele. This means that the method needs and considers passive internal reference substances and also respective positive controls, which must correspond to definite alleles. The internal controls are necessary to compensate for errors of the amplification conditions including those resulting from pipetting errors, inhibition of amplification, variable binding efficiencies of the probes, etc.; see U.S. Pat. No. 6,154,707 B1 and Shi, et al., Mol. Pathol. 52 (5) 295:-9 (1999): High throughput genotyping for the detection of a single nucleotide polymorphism in NAD(P)H quinone oxidoreductase (DT diaphorase) using TaqMan probes.

The present invention makes use of a novel and universal method for allele discrimination, which method is eminently suitable for detection of specific nucleic acid sequences, especially for determination of SNPs or other changes of nucleic acid sequences (such as mutations). The inventive method does not need any real-time instrumental systems but instead needs a thermocycler, which is known in itself, and a fluorescence-measuring instrument. The purchase price of instruments for this purpose is therefore much lower than the price needed for a real-time instrument.

For this purpose the inventive method does not make use of the internal controls or reference samples needed in U.S. Pat. No. 6,154,707 B1 and in Mol. Pathol. (1999) 52 (5) 295:-9 (AD-TaqMan®-PCR-Assay). As was described in Mol. Pathol. (1999) 52 (5) 295:-9 (page 297), the assay requires calibration steps for the already genotyped homozygous wild-type and mutant probes used as standards.

In contrast, the inventive method does not need any optimization steps or DNA standard samples. The primers and probes can be prepared in any desired concentration, which is not possible in the AD-TaqMan® PCR assay. Since the inventive method does not need any controls, it is capable of discriminating those alleles which very seldom carry mutations and for which no homozygous mutant genotypes are present.

In the AD-TaqMan® PCR assay, the comparison with the negative controls serves merely as contamination control. In the inventive method, allele discrimination is based on the measurements of the negative controls as will become evident from Example 2. A further advantage in principle of the inventive methodology compared with the known method consists in the freedom of choice of probes. All publications about the TaqMan® PCR technology advise against providing reporter dyes with a guanine molecule or placing such a molecule in their proximity. The probes should not have a G/C content higher than 80%. In the inventive method, these limitations do not exist.

The method can be used for numerous commercially available probe systems that are functionally completely different, which systems are used for PCR hybridization methods and are based on fluorescence resonance energy transfer (FRET) or other fluorescence-detection systems. Commercially available probe systems such as TaqMan™, Uniprimer™, TripleHyb™, Scorpions™, Molecular Beacons or terbium chelate probes can be used. From this it also results that different detection technologies may also be used and analyzed by means of the method, an example being the application of TaqMan probes in an exonuclease assay or the use of allele-specific double-labeled primers in a standard PCR reaction.

The detection reactions take place in two steps. The target-specific amplification reaction, coupled with the probe-hybridization reaction or with the use of fluorescence-labeled allele-specific amplification primers, takes place in a first step. This reaction can be performed on all commercial PCR instruments and is not limited to real-time PCR instruments. Fluorescence detection is then also accomplished on commercial fluorescence readers.

Preferably the inventive method makes use of the combination of a SpeedCycler (Analytik Jena AG) and of a fluorescence reader (SpeedScan: Analytik Jena AG). The advantage of this combination of instruments is based on the patented and extremely fast amplification technology (patent) by means of the SpeedCycler, which makes it possible to complete the amplification/hybridization reaction in approximately 30 minutes. The above products and methods are hereby incorporated by reference to U.S. Pat. Nos. 5,841,136; 5,939,312; 6,556,940; and 7,160,180.

The reaction mixtures contain, for example, inherently needed reagents, which are also used for real-time PCRs.

For each mixture, a statistically relevant number (e.g., from 3, 4, 5, 6, 7, 8, 9, 10 or more) of DNA-free negative controls is run in parallel. On the basis of these control samples it is possible on the one hand to ensure that the mixture is free of contamination, while on the other hand providing the samples that are needed for the calculation.

To determine whether amplification was successful or to check for the presence of contamination, the signal intensities of the samples are compared with the signal intensities of the negative controls. This is done in order that the samples for which amplification was not successful can be excluded from the subsequent evaluation. If the signal intensity of the negative controls is as strong as the signal intensity of the samples, then contamination of the samples is present. In this way the known problem of sample contamination in PCR-based methods is also taken into consideration.

Genotyping (allele discrimination) takes place by determining the ratio (K) of the end-point fluorescence of probe 1 (for example, labeled for allele 1 or for the wild-type sequence) and of probe 2 (for example, labeled for allele 2 or a nucleic acid sequence different from the wild-type sequence). This measured value is therefore completely independent of the known potential fluctuations of PCR conditions and efficiencies of probe binding.

The determination of the mean value of the ratio (K) for the negative controls yields information on whether or not a homozygous genotype containing allele 1 or a homozygous wild-type genotype is present, or in other words whether the samples are homozygous or heterozygous.

The comparison of each individual sample with the maximum value and minimum value of the ratios of all measured results as well as with the mean value of the negative controls yields information on whether the sample is homozygous for allele 1 or homozygous for the wild type or is heterozygous (presence of allele 1 and allele 2) or is a homozygous mutation.

In this way the inventive method permits the planned characterization of specific nucleic acid sequences simply, rapidly and reproducibly, especially in view of the detection of mutations or of the genotyping of SNPs.

The evaluation of the end-point fluorescence values can be achieved in the form of a computer program. In the process, statistical variables (such as the standard deviation) can be easily determined. Those skilled in the art are familiar with the determination of standard deviations.

The inventive method does not need any internal reference samples as positive controls or as controls for calculation of the fluorescence signals to be evaluated.

The inventive method permits precise assay of mutations or genotyping of SNPs, merely by taking the measured fluorescence maxima and fluorescence minima into consideration.

The inventive method can be applied to numerous assay formats being used on the basis of generation of fluorescence signals. In particular, the TaqMan probes are very suitable for the purpose of detection. Surprisingly, the inventive method circumvents an existing limitation of the application of TaqMan probes.

An essential aspect for the choice and design of TaqMan probes is that the reporter dye is not permitted to be bound to guanine, since the fluorescence of the probe is quenched even after exonuclease digestion. Furthermore, according to U.S. Pat. No. 6,154,707 B1, the GC content of the probes to be chosen should range between 20% and 80% and the melting temperature of the probe should not be higher than 70° C. By means of the inventive method, it is possible, for example, to choose TaqMan probes whose GC content is higher than shown in U.S. Pat. No. 6,154,707 B1. Such probes may have a GC content of 80%, >80%, 82.5%, 85%, 87.5%, 90% or more, including all intermediate values and subranges between 80% and 100% GC content. Moreover, the probe melting temperature may be 70° C., >70° C., 72.5° C., 75° C., 77.5° C., 80° C., or more, including all intermediate values and subranges. Furthermore, the reporter dye can even be coupled to a guanine, without negatively influencing an evaluation of the measured signals by fluorescence quenching. The sensitivity of the inventive method is therefore much higher than that of the method disclosed in the foregoing patent document.

The goal of the inventive method, which is to achieve detection of mutations or the genotyping of SNPs with the least possible experimental time and effort, can be surprisingly achieved by further simple methods or means. In this way the complex experimental method steps, which are included in an all-encompassing total process in the modern methods of molecular diagnostic tests, are taken into account.

Those skilled in the art are aware that assaying hereditary mutations, for example, or genotyping of SNPs is based on the following steps:

1. isolation of the genomic DNA of the test subject (for example from swabs of oral mucous tissue).

2. amplification/detection of the sequence portions to be examined.

For this purpose numerous working steps are necessary, and in particular the reaction mixtures for the amplification/detection reactions require pipetting of numerous reaction components.

According to the invention, a test kit is provided in order to simplify these working steps.

Those skilled in the art are aware that modern methods of isolation of genomic DNA, such as swabs of oral mucous tissue, are based on a procedure in which the nucleic acid to be isolated is bound on a solid mineral phase, washed and ultimately detached from the mineral phase with a low salt buffer (such as Tris HCl) or water. The detachment of the bound nucleic acids under these conditions falls within the prior art (for example, DE 41 39 644 A1, U.S. Pat. No. 5,234,809 B1, WO A 95/34569 B1, EP 1 135 479 A1, or DE 43 21 904 A1).

The nucleic acids present in these elution buffers are then used for the subsequent analysis process.

In the amplification/detection reactions that are necessary for the described molecular biological detection method, the reaction mixtures are then pipetted. This means that different reaction buffers as well as components and the isolated test subject DNA to be examined are brought together. For example, primers, fluorescence-labeled probes, dNTPs, amplification buffers, magnesium chloride, polymerase and possibly further additives are needed.

After these components have been brought together in exact quantitative proportions, the detection reaction can be started.

The inventive method makes use of very simple means, in order to minimize the steps needed to set up the amplification/detection reactions.

Surprisingly, it is found that the elution of nucleic acids bound on a solid mineral phase can be achieved with an elution buffer whose composition consists of salts, including Mg²⁺ ions and if necessary further additives such as BSA (bovine serum albumin), and permits it to be used directly in subsequent amplification/detection reactions. Such an elution buffer permits efficient elution of the bound DNA and thus has the fortuitous result that the components needed for the subsequent amplification/detection reactions (amplification buffer, Mg²⁺ ions, further PCR additives) do not have to be pipetted separately. Thus the molar composition of the inventive elution buffer can be adjusted such that dilution can be subsequently achieved with water or such dilution is even made unnecessary, in which case the eluting agent can be used directly in the detection reaction.

The dual functionality of the inventive elution buffer thus permits highly efficient elution and a savings of time and effort for the necessary preparation of the reaction mixture for amplification/detection reactions.

A further simplification is achieved by converting the further reaction components that will still be needed, such as dNTPs, primers, possibly probes and polymerases, into a storage-stable form. Those skilled in the art are aware that reaction mixtures can be converted to storage-stable condition by means of lyophilization, for example, and that such a mixture can be reactivated by addition of an aqueous phase. However, it is also known that this type of stabilization is not always free of problems and needs considerable expenditure for apparatus. Furthermore, especially in lyophilization of reaction mixtures, problems exist with excessive drying, as a consequence of which the biological activity of enzymes is no longer restored or is not fully restored.

This also pertains to processes of direct drying by heating, for which purpose high temperatures are generally used. Such a mixture is naturally based on the knowledge that, for example, the biological activity of proteins is contingent upon the presence of water. From this it is concluded that stabilization of biologically active reaction mixtures can be achieved by rapid removal of water. This means that high temperatures are always used for drying of reaction mixtures, to ensure that this process will be accomplished in a short time. Surprisingly, however, it is found that the removal of water from reaction components to be stabilized (polymerase, dNTPs, primers and possibly probes) also functions at physiological temperatures, and as a result needs longer times.

Using the inventive simple drying method at physiological temperatures (30° C. to 40° C.), it is therefore possible to prepare a functionally active plastic reaction substrate with simple means (such as a drying oven). This plastic substrate can then be stored for months at ambient temperatures without loss of reaction efficiency.

In the combination of a novel elution buffer in the process of DNA isolation, and by the use of the inventively modified plastic substrate with the components needed for the amplification/detection reactions, the invention ultimately permits an extreme simplification of the time and effort for pipetting. After elution of the DNA, a definite volume of elution buffer is now merely transferred into a plastic substrate that has been modified to storage-stable form and contains the further specific reaction components, and the amplification/detection reaction is started.

This simplification—in combination with the inventive method for allele discrimination—therefore permits routine, simple and extremely rapid performance of molecular diagnostic tests for detection of mutations and for SNP genotyping. A particularly efficient alternative embodiment consists in the use of a SpeedCycler (Analytik Jena AG) for the extremely fast amplification/detection reaction and the subsequent measurement on a commercially available fluorescence reader (SpeedScan; Analytik Jena AG). By virtue of the extremely fast amplification reactions, the use of this combination of instruments allows the tests to be carried out in an extremely short time. All needed steps, from DNA isolation using the inventive elution buffer, the use of the storage-stabilized plastic reaction substrate for the amplification/detection reaction and the performance of these reactions on a SpeedCycler/SpeedScan and the integration of the evaluation algorithm in a computer-assisted software solution, permit the determination of mutations or of SNPs in less than one hour. In this connection, as many as 96 samples can be analyzed, depending on the plastic reaction substrate. All necessary handling steps are reduced to a minimum. Furthermore, because only very small amounts of reaction chemistry are now needed, the assays to be performed are also much less expensive than the tests that can currently be performed on the real-time systems already described in detail. In this way the present invention can make a valuable contribution to the routine establishment of molecular genetic tests, especially in individualized medicine, which is attracting increasing attention.

The present invention will be explained in more detail hereinafter on the basis of the examples. Those examples do not represent any limitation of the application of the invention.

### EXAMPLES

### Example 1

Preparation of plastic reaction substrate having storage-stable coating, and use of the plastic substrate in a storage test on amplification of a human-specific target sequence

A. Preparation of the Coated Plastic Reaction Substrate

36-well microplates (Analytik Jena AG) were used as the plastic substrate.

The following components were added to each well:
1. Ligand-modified hot-start Taq DNA polymerase
2. dNTPs (deoxyribonucleotides)
3. Primers (sense and antisense)

The dead volume of the reaction mixture was 2.5 μL.

The coating solution was applied on the PCR microplate and dried for approximately 2 hours under physiological temperature conditions of 37° C.

After completion of drying, the plates were masked with a sealing film, stored at RT for 4 months and tested with freshly added reaction components in a comparison reaction.

B. Amplification of a Human-Specific Target Sequence

The amplification reaction was carried out by means of a SpeedCycler (Analytik Jena AG).

Mixtures:

“Fresh”:
1 μL DNA (human)
1.5 μL amplification buffer, including magnesium chloride (10×)
0.3 μL dNTPs (12.5 mM in each case)
0.1 μL primer (sense; 50 pmol)
0.1 μL primer (antisense; 50 pmol)
0.75 units of Taq polymerase
Make up with water to final volume of 15 μL.

“Microplates with storage-stable coating”:
1 μL DNA (human)
1.5 μL amplification buffer, including magnesium chloride (10×)
Make up with water to final volume of 15 μL.

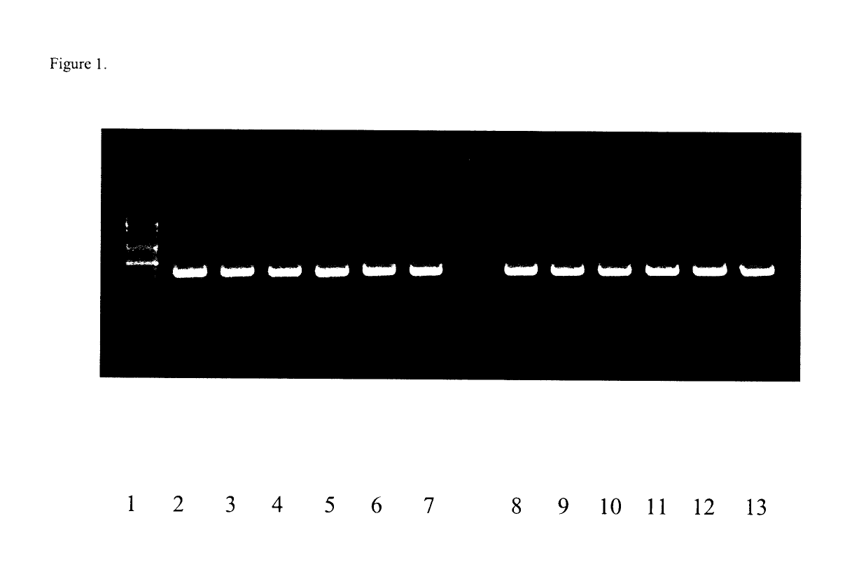

After the amplification reaction was carried out by means of a SpeedCycler, the amplification products were evaluated on an agarose gel (FIG. 1).
Lane 1: DNA marker
Lanes 2-7: Amplification products using the plastic reaction substrate with storage-stable coating
Lanes 8-13: Amplification products using freshly added reaction components

The example illustrates that the use of the reaction product with storage-stable coating does not exhibit any loss of activity compared with freshly added reaction components.

### Example 2

SNP genotyping by means of the inventive method, and comparison of the results by means of DNA sequencing. The inventive method was then used for SNP genotyping of human DNA samples. The results obtained with the inventive method were then sequenced for verification. The results of sequencing and of genotyping by means of the inventive method are presented in summary form.

A mutation in the promoter of the human MDM2 gene (SNP 309) [Bond GL et al., A single nucleotide polymorphism in the MDM2 promoter attenuates the p53 tumor suppressor pathway and accelerates tumor formation in humans. Cell. 2004 Nov. 24; 119(5):591-602] was chosen for genotyping.

The amplification/detection reaction of the sample nucleic acids used was carried out by means of the SpeedCycler (amplification/hybridization) and SpeedScan (detection of end-point fluorescence) instruments of Analytik Jena AG.

The following oligonucleotides were used as primers for amplification:

| | |
|---|---|
| Sense primer: | |
| 5′-CGC GGG AGT TCA GGG TAA-3′ | (SEQ ID NO: 1) |
| Antisense primer: | |
| 5′-CCC AAT CCC GCC CAG ACT AC-3′ | (SEQ ID NO: 2) |

The following doubly fluorescence-labeled nucleotides were used as hybridization probes, the GC content for both probes being >80% and the melting temperature being higher than 70° C.:

Wild-type probe:

(SEQ ID NO: 3)

5′-FAM-GGG CCG CTT CGG CGC GGG-BHQ1-3′
Mutated probe:

(SEQ ID NO: 4)

5′-ROX-GGC CGC TGC GGC GCG-BHQ2-3′

The amplification/hybridization reaction needed approximately 35 minutes for 40 completed amplification cycles.

For 36 samples, detection needed approximately 2 minutes in each case.

As an example for all 3 possible allele variants, the genotyping by means of the inventive method is listed hereinafter.

The fluorescence of the samples for labeling probe 1 (FAM) and probe 2 (ROX) was measured with the SpeedScan end-point fluorescence reader (Analytik Jena AG). The measured results are presented in the following table:

| Sample No. | FAM fluorescence | ROX fluorescence |
|---|---|---|
| 2 (WT) | 2,210 | 1,442 |
| 3 (heterozygous) | 1,342 | 1,449 |
| 5 (homozygous mutant) | 1,131 | 1,999 |
| Negative control (mean value) | 542 | 642 |
| Standard deviation of the | 63.6 | 104.9 |
| neg. cont. | | |

First, the necessary statistical variables for the measurement plate were calculated:

Step 1: Determination of whether amplification was successful and checking whether contamination is present.

| | | |
|---|---|---|
| For sample 2: | FAM | 2,210 > 542 + 63.6 |
| | ROX | 1,442 > 642 + 104.9 |
| For sample 3: | FAM | 1,342 > 542 + 63.6 |
| | ROX | 1,449 > 642 + 104.9 |
| For sample 5: | FAM | 1,131 > 542 + 63.6 |
| | ROX | 1,999 > 642 + 104.9 |

The results confirmed that successful amplification took place in each of the three samples.

Step 2: Genotyping (allele discrimination)

1. Calculation of the ratio (K) between the measured results of wild-type probe 1 (FAM) and mutant probe 2 (ROX). As shown below, higher amounts of wild-type probe (FAM) bound to wild-type DNA (Sample 2) than either heterozygous or homozygous mutant targets in Samples 3 and 5.

| Sample No. | Ratio K |
|---|---|
| 2 (WT) | 1.53 (2,210/1,442) |
| 3 (heterozygous) | 0.93 (1,342/1,449) |
| 5 (homozygous mutant) | 0.56 (1,131/1,999) |
| Mean value of the negative controls | 0.85 (542/642) |
| Maximum value | 1.54 |
| Minimum value | 0.565 |

2. Comparison of the maximum value (MAX) with the mean value of the negative controls adjusted by the standard deviation of the negative controls showed that the samples with the homozygous wild-type genotype are present on the plate, because the maximum value is greater than the mean value of the negative controls adjusted by the standard deviation.

3. Determination of the minimum value (MIN) for the ratio of all measured results.

MIN=

4. Comparison of the minimum value (MIN) with the mean value of the negative controls minus the standard deviation of the negative controls showed that the samples with the homozygous mutant genotype are present on the plate, because the minimum value is smaller than the mean value of the negative controls adjusted by the standard deviation.

5. Comparison of each individual sample with the maximum value, minimum value and mean value of the negative controls.

For sample 2: Comparison of the mean value of the negative controls with the maximum value shows that sample 2 is homozygous wild-type and that further calculations for sample 2 are unnecessary, since the difference between the mean value of the negative controls and ratio K of sample 2 is larger than the difference between the maximum value and ratio K of sample 2 adjusted by the standard deviation.

For sample 3: Sample 3 can in no case have a homozygous wild-type genotype, and the possibility that it belongs to the two other genotype groups must be checked, since the difference between the maximum value and ratio K of sample 3 adjusted by the standard deviation is larger than the negative controls and ratio K of sample 3.

For sample 5: Sample 5 can in no case have a homozygous wild-type genotype, and the possibility that it belongs to the two other genotype groups must be checked, since the difference between the maximum value and ratio K of sample 5 adjusted by the standard deviation is larger than the negative controls and ratio K of sample 5.

Further examination of samples 3 and 5:

For sample 3: Comparison of the mean value of the negative controls with the minimum value shows that sample 3 can in no case have a homozygous mutant genotype, since this was already calculated above, and also that it is not of homozygous wild type but can only be heterozygous, since the difference between the mean value of the negative controls and ratio K of sample 3 is smaller than the difference between the minimum value and ratio K of sample 3 adjusted by the standard deviation.

For sample 5: Comparison of the mean value of the negative controls with the minimum value shows that sample 5 has a homozygous mutant genotype, since the difference between the mean value of the negative controls and ratio K of sample 5 is larger than the difference between the minimum value and ratio K of sample 5 adjusted by the standard deviation. In this way the genotypes of the samples were defined. The calculation described in the foregoing was performed in a logical process that is suitable for a computer algorithm.

Comparison of Genotyping by the Method of the Invention and by Sequencing.

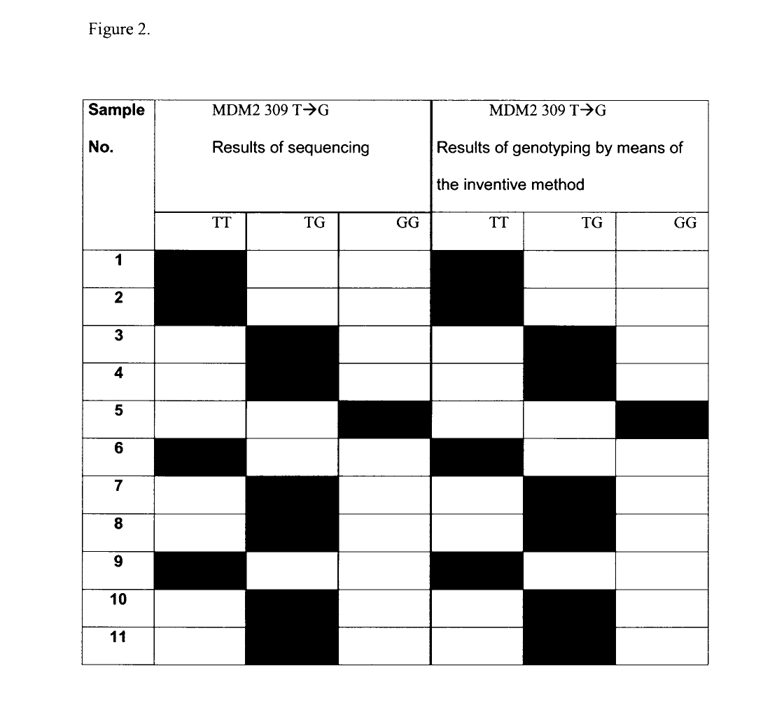

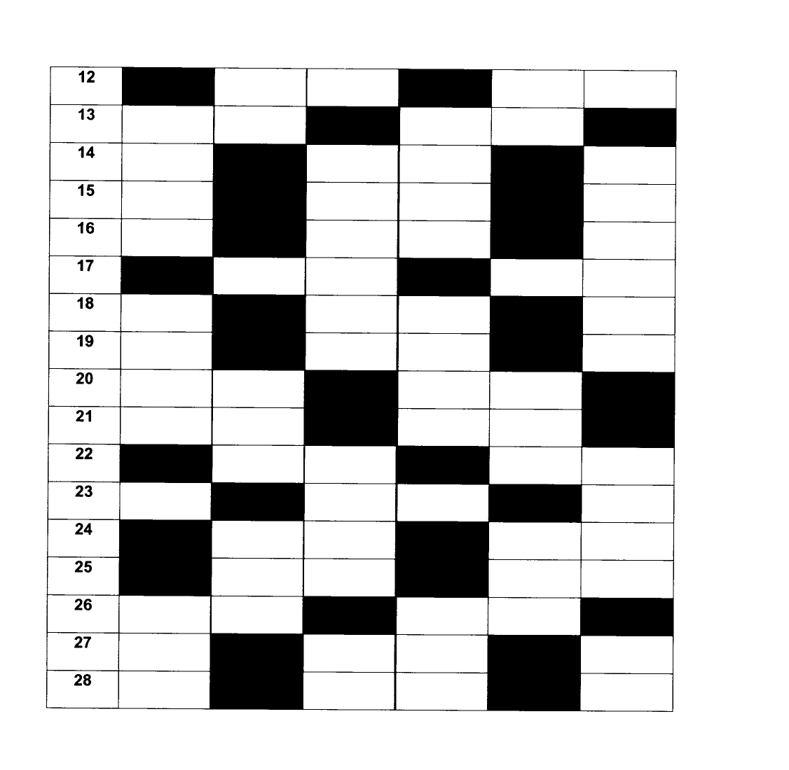

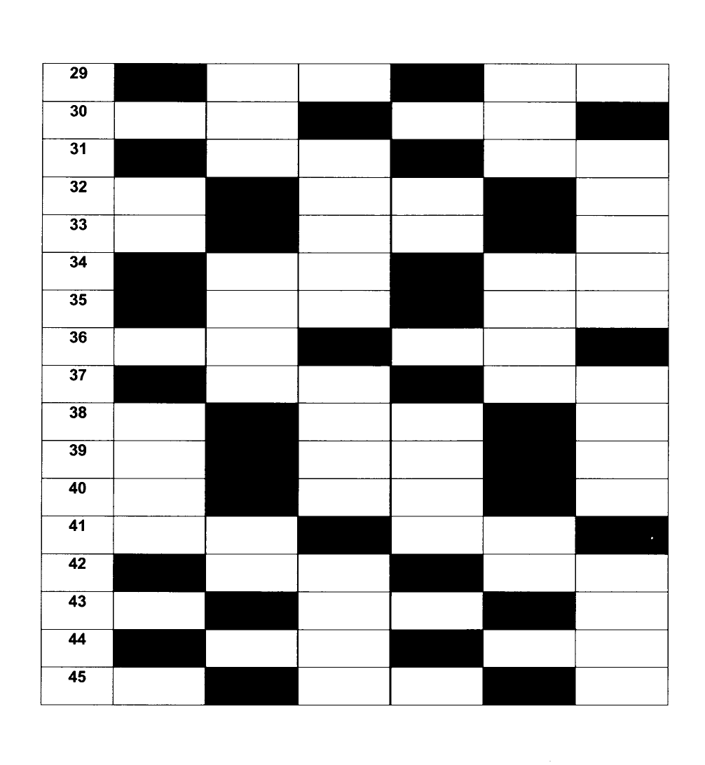

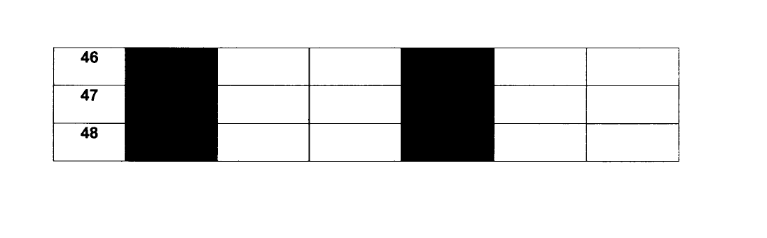

To check the reproducibility of the inventive methodology, genotyping was carried out for 48 different sample nucleic acids. The results obtained by means of the inventive methodology were checked by means of sequencing. The comparison, shown in FIG. 2, demonstrates that the method of the invention identified the genotype of each sample in the same way as sequencing.

### Example 3

Genotyping of genetic Factor V_{Leiden} (G1691A: Alhenc-Gelos M. et al., Unexplained thrombosis and factor V Leiden mutation. Lancet; 1994) by means of the inventive method

Genotyping was carried out by means of the inventive method as follows:

1. DNA isolation from oral mucus tissue swabs using the inventive elution buffer. The swabs were isolated as follows by means of a commercially available DNA extraction kit (innuPREP DNA Mini Kit; AJ Innuscreen GmbH). The kit is based on lysis of the starting material, subsequent binding of the DNA on the surface of a filter material (spin filter column), washing of the bound DNA and finally elution of the DNA by means of water or by means of a solution containing 10 mM Tris HCl. Instead of the eluting agent contained in the kit, the inventive elution buffer was used (33 mM tricine-KOH, 14 mM KCl, 4.2 mM MgCl₂, 3.5 μg/mL BSA; pH 9). The DNA was eluted after addition of 500 μL of the eluting agent from the spin filter column. The eluted DNA was then used directly for the amplification/detection reaction.

2. Amplification/detection reaction using the plastic substrate with storage-stable coating. The plastic reaction substrate was prepared as described in Example 1 and coated with the following components:
1. Ligand-modified hot-start Taq DNA polymerase
2. dNTPs
3. Primers (sense and antisense)

The coating solution was applied on the PCR microplate and dried for approximately 2 hours under physiological temperature conditions of 37° C.

The following oligonucleotides were used as primers for amplification:

Sense primer:

(SEQ ID NO: 5)

5′-GCC TCT GGG CTA ATA GGA CTA CTT C-3′
Antisense primer:

(SEQ ID NO: 6)

5′-TTT CTG AAA GGT TAC TTC AAG GAC AA-3′

10 μL of the DNA eluted by means of the novel elution buffer was transferred directly onto the coated PCR plates. Thereafter the only other reaction components added were the fluorescence-labeled hybridization probes.

The following doubly fluorescence-labeled nucleotides were used as hybridization probes:

Wild-type probe:

(SEQ ID NO: 7)

5′-FAM-ACC TGT ATT CCT CGC CT-BHQ1-3′
Mutated probe:

(SEQ ID NO: 8)

5′-ROX-ACC TGT ATT CCT TGC CT-BHQ2-3′

After completion of the amplification/detection reaction by means of SpeedCycler and SpeedScan (Analytik Jena AG), the measured end-point fluorescence was evaluated by means of the inventive method. The measured results are presented in the following table:

| Sample No. | FAM fluorescence | ROX fluorescence |
|---|---|---|
| 1 | 8,967 | 815 |
| 2 | 5,129 | 1,239 |
| 3 | 7,052 | 854 |
| 4 | 10,450 | 899 |
| 5 | 2,798 | 4,351 |
| 6 | 4,702 | 1,046 |
| Negative control (mean value) | 1,564 | 874 |
| Standard deviation of | 256.5 | 55.7 |
| the neg. cont. | | |

After the calculation described in Example 2 had been performed, it was possible to genotype the samples as follows:

MW_{neg}=1.79 STDEV_{neg}=0.19

| | Ratio of | MAX | MIN | MW_{neg}- | |
|---|---|---|---|---|---|
| Sample | probe 1/probe 2 | sample | sample | sample | Genotype |
| 1 | 11 | 0.6 | 10.36 | 9.21 | Wild type |
| 2 | 4.1 | 7.5 | 3.46 | 2.31 | Heterozygous |
| 3 | 8.3 | 3.3 | 7.7 | 6.51 | Wild type |
| 4 | 11.6 | 0 | 10.96 | 9.81 | Wild type |
| 5 | 0.64 | 10.96 | 0 | 1.15 | Homozygous |
| | | | | | mutant |
| 6 | 4.5 | 7.1 | 3.86 | 2.71 | Heterozygous |

The entire method now permits genotyping of multiple samples in less than one hour, beginning with isolation of the test subject DNA until final evaluation.

### Modifications and Other Embodiments

Various modifications and variations of the described products, methods and kits as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the molecular biological, medical, biological, chemical or pharmacological arts or related fields are intended to be within the scope of the following claims.

### INCORPORATION BY REFERENCE

Each document, patent, patent application or patent publication cited by or referred to in this disclosure is incorporated by reference in its entirety, especially with respect to the specific subject matter surrounding the citation of the reference in the disclosure. However, no admission is made that any such reference constitutes background art and the right to challenge the accuracy and pertinence of the cited documents is reserved.

1. A method for rapidly detecting a specific target nucleic acid sequence, detecting a mutation, or detecting an SNP in an isolated nucleic acid sample, comprising:
(i) amplifying a specific target sequence and hybridizing it to fluorescence-labeled allele-specific amplification probes;
measuring the endpoint fluorescence of the hybridized nucleic acid formed by each probe; and
determining the genotype of the specific target sequence from the ratio of fluorescence of the two probes;
wherein the endpoint fluorescence is determined without the use of reference dyes and without comparison with a previously genotyped standard sample or internal control; and
wherein the endpoint fluorescence measurement is determined after elimination of contaminated samples as determined from parallel negative controls that contain the probes but do not contain the specific target sequence.

2. The method of claim 1, wherein each fluorescence-labeled allele-specific amplification probe is labeled with a different fluorescent agent.

3. The method of claim 1, further comprising isolating the nucleic acid sample prior to amplifying the target sequence.

4. The method according to claim 1, wherein the isolated nucleic acid sample is contained in the same buffer as that used for amplification of the specific target sequence.

5. The method according to claim 1, wherein the DNA is obtained from a human.

6. The method according to claim 1, wherein the evaluation of the end-point fluorescence values and subsequent determination of the genotyping is performed using computer software.

7. The method according to claim 1, wherein the amplification is performed on a thermocycler and the measurement of the end-point fluorescence is measured using a fluorescence reader.

8. The method according to claim 1, wherein for each mixture, a statistically relevant number of DNA-free negative controls is run in parallel.

9. The method according to claim 1, wherein the genotyping takes place from the ratio of the end-point fluorescence of the samples and negative controls.

10. A nucleic acid amplification kit comprising:
a sense primer and an antisense primer capable of amplifying a target nucleic acid sequence, and
at least two different fluorescence-labeled oligonucleotides probes that respectively bind to a target nucleic acid sequence and to an allele of the target nucleic acid sequence.

11. The kit of claim 10, further comprising a nucleic acid amplification buffer.

12. The kit according to claim 10, further comprising a nucleic acid amplification buffer that contains tricine-KOH, bicine or Tris, at least one monovalent cation, and at least one divalent cation.

13. The kit according to claim 10, wherein the buffer contains BSA.

14. The kit of claim 10, further comprising a polymerase.

15. The kit of claim 10, further comprising a thermostable DNA polymerase.

16. The kit according to claim 10, further comprising reaction vessels on which polymerase, dNTPs and the primers are located in a storage-stable form.

17. The kit according to claim 10, wherein said probes are dual-sample probes (Taqman) in which the reporter dye is coupled to a terminal guanine.

18. The kit according to claim 10, wherein the GC content of the probes is greater than 80%.

19. The kit according to claim 10, wherein the GC content of the probes is greater than 85%.

20. The kit according to claim 10, wherein the melting temperature of the probes by melting point analysis is higher than 70° C.

21. The kit according to claim 10, wherein the melting temperature of the probes by melting point analysis is higher than 75° C.


SEQUENCE LISTING (8 sequences)

SEQ ID NO: 1 - DNA, 18 bp, Artificial Sequence
  Synthetic DNA; sense primer
cgcgggagttcagggtaa

SEQ ID NO: 2 - DNA, 20 bp, Artificial
  Synthetic DNA; antisense primer
cccaatcccgcccagactac

SEQ ID NO: 3 - DNA, 18 bp, Artificial
  Synthetic DNA; wild-type probe
gggccgcttcggcgcggg

SEQ ID NO: 4 - DNA, 15 bp, Artificial
  Synthetic DNA; mutated probe
ggccgctgcggcgcg

SEQ ID NO: 5 - DNA, 25 bp, Artificial
  Synthetic DNA; sense primer
gcctctgggctaataggactacttc

SEQ ID NO: 6 - DNA, 26 bp, Artificial
  Synthetic DNA; antisense primer
tttctgaaaggttacttcaaggacaa

SEQ ID NO: 7 - DNA, 17 bp, Artificial
  Synthetic DNA; wild-type probe
acctgtattcctcgcct

SEQ ID NO: 8 - DNA, 17 bp, Artificial
  Synthetic DNA; mutated probe
acctgtattccttgcct